(19) United States
(12) Patent Application Publication
(10) Pub. No.: US 2009/0254121 A1
(43) Pub. Date: Oct. 8, 2009

(54) Vascular Closure with Multi-Pronged Clip

(75) Inventors: Joshua M. O. Newth, Palo Alto, CA (US); Bernard A. Hausen, Redwood City, CA (US); Bryan D. Knodel, Flagstaff, AZ (US)

Correspondence Address: CARDICA, INC., 900 SAGINAW DRIVE, REDWOOD CITY, CA, 94063, US

(73) Assignee: CARDICA, INC., Redwood City, CA (US)

(21) Appl. No.: 12/061,350

(22) Filed: Apr. 2, 2008

(51) Int. Cl. A61B 17/064

(52) U.S. Cl. 606/219

(57) ABSTRACT

A medical device for closing an opening in tissue may include a staple including a plurality of tines, where the staple is deformable from an initial configuration to a splayed configuration and then a closed configuration and a shuttle fixed to the staple, where the shuttle includes a weakened area proximal to the shuttle. A method for closing an opening in tissue of a patient with that medical device may include moving the staple to a location in proximity to the opening; plastically deforming the staple to a splayed configuration; penetrating at least one of the tines into tissue in proximity to the opening; plastically deforming the staple to a closed configuration; and fracturing said shuttle at the weakened area, whereby the staple and a fragment of the shuttle fixed to the staple remain in the patient.

### FIELD OF THE INVENTION

The present invention relates generally to a system for closing an opening in tissue.

### BACKGROUND

Millions of people each year undergo catheterization for reasons including stent placement, angioplasty, angiography, atrial ablation, placement of abdominal aortic aneurysm grafts and/or stents, and other interventional cardiologic and vascular procedures. In a femoral artery catheterization, an opening is made in the wall of the femoral artery, and a sheath is placed in that opening through which a guidewire and one or more tools may be inserted for performing treatment on the patient.

After the sheath is removed, the opening in the femoral artery must be closed. Compression is typically used to do so. Anticoagulation therapy is stopped, and manual pressure is applied to the site for up to an hour until clotting seals the access site. The patient then must remain motionless for up to 24 hours, generally with a sandbag or other heavy weight on the site to continue the compression. Patients may find this procedure, and the resultant bruising and pain, to be more unpleasant than the actual interventional procedure that was performed.

Several types of closure devices and techniques have been developed in an attempt to facilitate closure of the opening in the femoral artery. However, acceptance of these devices and techniques has been limited for several reasons, including complexity of use, complication rates similar to traditional closure, and cost. One type of device utilizes suture to close the opening. However, such devices are typically complex mechanically and consequently are complex to operate. Further, such devices often require an auxiliary knot-pushing tool to be used, further increasing complexity. Other devices are ring-shaped or shaped in a convoluted or tortuous manner, and may be complicated and expensive to manufacture. Another closure technique involves inserting a plug or slurry of collagen or other chemical composition into the opening and/or the pathway in the leg between the opening and the skin. However, compression and lengthy bed rest are generally still required with chemical closure techniques, just as with traditional closure.

### BRIEF DESCRIPTION OF THE DRAWINGS

FIG. 1 is a schematic view of a vascular closure system that includes an end effector, a shaft and a handle.

FIG. 2 is a schematic view of tissue having a catheterization sheath positioned therein.

FIG. 3 is a perspective view of the end effector having butterfly members in a first, collapsed configuration.

FIG. 3A is a cross-section view of a butterfly member of the end effector along the line A-A in FIG. 3.

FIG. 3B is a cross-section view of a butterfly member of the end effector along the line B-B in FIG. 3.

FIG. 4 is a side view of the distal end of one exemplary butterfly member.

FIG. 5 is a side view of the distal end of a different exemplary butterfly member.

FIG. 6 is a perspective view of the end effector having exemplary butterfly members in a second, expanded configuration.

FIG. 7 is a top view of the end effector of FIG. 6.

FIG. 8 is a side view of an optional spring connected to a butterfly member.

FIG. 9 is an exploded view of an exemplary end effector.

FIG. 10 is a perspective view of an exemplary staple.

FIG. 11 is a different perspective view of the staple of FIG. 10.

FIG. 12 is a side view of the staple of FIGS. 10-11 in a first configuration, connected to a shuttle.

FIG. 13 is a perspective cutaway view of the exemplary end effector of FIG. 9, in a first configuration.

FIG. 14 is a perspective view of a splay arm.

FIG. 15 is a perspective view of the exemplary end effector of FIG. 9, in a first configuration, with one butterfly member omitted for clarity.

FIG. 16 is a side view of a first step in the operation of the closure system.

FIG. 17 is a perspective view of the end effector having butterfly members in a second, expanded configuration.

FIG. 18 is a side view of a second step in the operation of the closure system.

FIG. 19 is a side view of a third step in the operation of the closure system.

FIG. 20 is a side view of a fourth step in the operation of the closure system.

FIG. 21 is a side view of a fifth step in the operation of the closure system.

FIG. 22 is a perspective view of the end effector after the butterfly members have been moved proximally.

FIG. 23 is a perspective cutaway view of the exemplary end effector of FIG. 9, in a second configuration, with one butterfly member omitted for clarity.

FIG. 24 is a side view of the second configuration of FIG. 23.

FIG. 25 is a detail perspective view of the distal end of a driver.

FIG. 26 is a perspective cutaway view of the exemplary end effector of FIG. 9, in a third configuration, with one butterfly member omitted for clarity.

FIG. 27 is a detail perspective view of a tip sleeve of the end effector, showing tabs defined therein.

FIG. 28 is a perspective cutaway view of the exemplary end effector of FIG. 9, in a fourth configuration, with one butterfly member omitted for clarity

FIG. 29 is a side view of the closed staple, with a fragment of the shuttle attached thereto.

The use of the same reference symbols in different figures indicates similar or identical items.

### DETAILED DESCRIPTION

Closure System

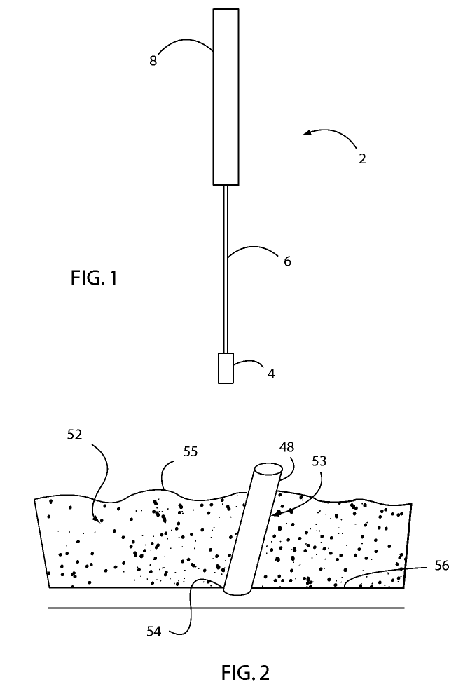

Referring to FIG. 1, a closure system 2 includes an end effector 4 connected to a tool shaft 6, which in turn is connected to a handle 8. The end effector 4 may include one or more separate components that are fabricated separately and then connected to the tool shaft 6, or may be fabricated integrally with the distal end of the tool shaft 6. The handle 8 may be configured in any manner that allows for actuation of the end effector 4. Referring also to FIG. 2, the end effector 4 is sized to pass through a standard sheath 48 placed in a passage 53 in tissue 52 for a standard catheterization procedure. The end effector 4 may include a housing 22 configured in any suitable manner.

Butterfly Members

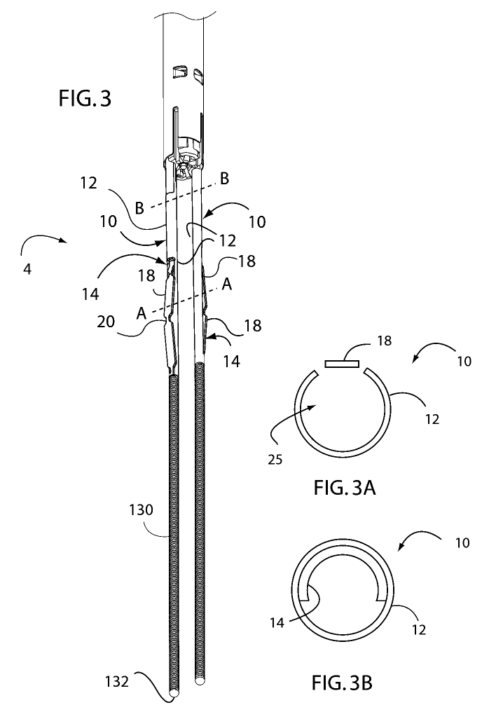

Referring also to FIG. 3, the end effector 4 may include at least one butterfly member 10. Each butterfly member 10 acts to register tissue such as the wall of a blood vessel to the end effector 4, as described in greater detail below. At least one butterfly member 10 may extend substantially distally from the end effector 4. Alternately, at least one butterfly member 10 may extend at least partially in a different direction, or may be positioned outside of or separate from the end effector 4. Each butterfly member 10 may be configured in any manner that allows it to move from a first, collapsed configuration to a second, expanded configuration, and back to the collapsed configuration. As one example, at least one butterfly member 10 includes a first element 12, and a second element 14 connected to the distal end of the first element 12. The first element 12 is not substantially deformable, and at least part of the second element 14 is deformable to an expanded configuration. Alternately, either or both of the elements 12, 14 is deformable to an expanded configuration. The elements 12, 14 may be shaped and configured in any suitable manner. As one example, referring also to FIGS. 3A-3B, the first element 12 may have a semicircular cross-section or other curved cross-section along at least part of its length. Such a cross section increases the moment of inertia of the first element 12 and thereby increases its stiffness. At least part of the first element 12 may be partially tubular, hollow, or otherwise include an area configured to receive a portion of the second element 14, or vice versa. At least part of the first element 12 may be substantially coaxial with the second element 14. The distal end 16 of at least one butterfly member 10 may be blunt in order to prevent or minimize any disturbance to the tissue structure into which the butterfly member 10 is inserted. For example, the distal end of at least one butterfly member 10 may be curved at the junction between the elements 12, 14. At least one butterfly member 10 may have a longitudinal axis in the collapsed configuration that is offset from and substantially parallel to the longitudinal axis of the end effector 4 and/or the tool shaft 6. The use of the term “axis” in this document is not limited to use with respect to structures that are cylindrical or radially symmetrical, and the use of the term “axis” in conjunction with a structure does not and cannot limit the shape of that structure. Alternately, at least one butterfly member 10 is oriented differently relative to the longitudinal axis of the end effector 4 and/or the tool shaft 6. The first element 12 may extend through the tool shaft 6 to the handle 8.

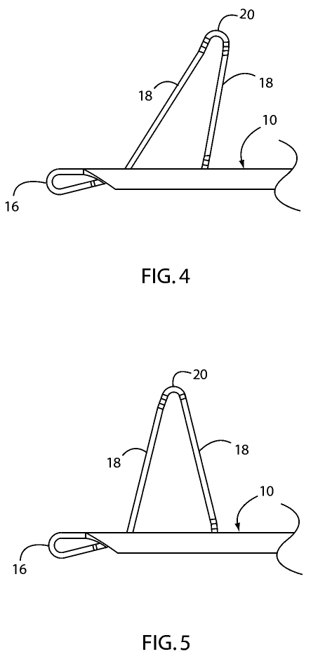

The second element 14 may include two substantially planar segments 18 longitudinally spaced from one another and connected to one another by a hinge element 20 between them, such that one segment 18 is positioned distal to the other segment. The segments 18 need not be planar. For example, at least one segment 18 may be curved. At least one segment 18 may have a radius of curvature substantially the same as a remainder of the second element 14. The hinge element 20 may be a living hinge, such as a narrower area between the two segments 18 that bends to allow movement between the segments 18. Alternately, the hinge element 20 may be any structure or mechanism that allows for relative movement between the segments 18. At least one of the segments 18 may be curved or otherwise non-planar. One of the planar segments 18 may extend to a location at or in proximity to the distal end 16 of the corresponding butterfly member 10. The segments 18 may be angled relative to one another when the butterfly member 10 is in the first, collapsed configuration. For example, the most-distal segment 18 may be angled relative to the longitudinal axis of the corresponding butterfly member 10 such that the distal end of that segment 18 is closer to that longitudinal axis than the proximal end of that segment 18, and the most-proximal segment 18 may be angled relative to the longitudinal axis of the corresponding butterfly member 10 such that the proximal end of that segment 18 is closer to that longitudinal axis than the distal end of that segment 18. Alternately, the segments 18 may be angled differently relative to one another. The angle between the segments 18 allows the hinge 20 to deform or otherwise move upon application of force to the second element 14, as described in greater detail below. The segments 18 may be angled relative to one another a greater amount when the butterfly member 10 is in the second, expanded configuration than in the first, collapsed configuration. Alternately, the segments 18 may be substantially parallel to one another and/or lie in substantially the same plane as one another. Referring to FIG. 5, the segments 18 each may be substantially the same length, such that they form a symmetrical shape upon actuation of the butterfly member 10. Alternately, referring to FIG. 4, the segments 18 may differ in length, such that they form a non-symmetrical shape upon actuation of the butterfly member 10. Thus, in the expanded configuration, the distal end of at least one butterfly member 10 may have a single expanded feature formed by the connected segments 18.

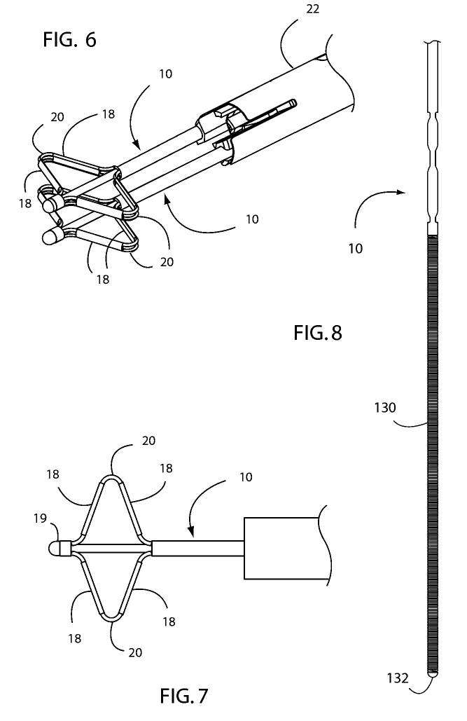

Alternately, referring to FIGS. 6-7, in the expanded configuration, the distal end of at least one butterfly member 10 may have more than one expanded feature formed by connected segments 18. That is, at or near the distal end of the second element 14 of at least one butterfly member 10, two or more sets of segments 18 may be provided. Each set of segments 18 may include two segments 18 connected by a hinge 20 that may include two or more separate elements. Alternately, the segments may be connected differently. As shown in FIGS. 6-7, two sets of segments 18 are provided on each butterfly member 10, spaced opposite one another near the distal end of the corresponding butterfly member 10. However, the two sets of segments 18 may be oriented differently relative to one another. Further, more than two sets of segments 18 may be provided on at least one butterfly member 10. The sets of segments 18 need not be arranged radially symmetrically or in any other particular arrangement relative to the remainder of the corresponding butterfly member 10. Each segment 18 in a set may be substantially the same length and/or shape as the other, or may differ in length and/or shape from the other.

The distal end of at least one butterfly member 10 may be coated, cushioned, shaped and/or otherwise configured to prevent or minimize any disturbance to the tissue structure into which the butterfly member 10 is inserted, as described in greater detail below. As one example, referring to FIGS. 6-7, a soft tip 19 may be located on the distal end of at least one butterfly member 10. The soft tip 19 may be fabricated from silicone or any other suitable biocompatible material. The soft tip 19 may be overmolded onto the distal end of the corresponding butterfly member 10, or attached in any other suitable manner, such as by adhesive. The soft tip 19 may be utilized in conjunction with any configuration of the butterfly member 10, including the configuration of FIGS. 3-5.

As another example of cushioning, referring to FIG. 8, a spring 130 may be attached to the distal end of at least one butterfly member 10. The spring 130 may extend any suitable length from the distal end of the corresponding butterfly member 10. The spring 130 may have a generally constant diameter, may taper extending in the distal direction, or may be shaped differently. Advantageously, the spring 130 is a coil spring, having any suitable diameter. As one example, the spring 130 may have a diameter slightly greater than that of the distal end of the corresponding butterfly member 10, such that the spring 130 may be pressure-fit onto, welded onto, connected with adhesive to, or otherwise connected to the outer surface of the corresponding butterfly member 10. A central core (not shown) may extend along the center of the spring 130. If so, the central core may be fabricated integrally with the spring 130 in any suitable manner. The distal end of the spring 130 and/or the central core may be fixed to a cap 132 that may have a width at least as great as the distal end of the spring 130. The cap 132 may be a soft tip such as the soft tip 19 described above. As another example, the cap 132 may be a hemispherical component, where the rounded portion of that component is oriented distally, that is hard, smooth, and attached to the spring 130 in any suitable manner, such as by welding, by adhesive, by pressure fitting, or by any other suitable mechanism, structure or method. The cap 132 may be shaped differently, if desired. Optionally, the cap 132 may apply compressive force to the spring 130.

A proximal portion of the second element 14 may extend into a center area 25 of a proximal portion of the first element 12. That center area 25 of the first element 12 may be referred to as the lumen of the first element 12 for convenience, even though the first element 12 may be open along part of its perimeter, or may have a cross-section other than circular, at any portion of its length. The longitudinal axis of the lumen 25 may be substantially coincident with the longitudinal axis of the first element 12, or may be offset from or otherwise aligned relative to the longitudinal axis of the first element 12. The second element 14 may be movable relative to that lumen 25, such as by sliding substantially along or substantially parallel to the longitudinal axis of the lumen 25. Alternately, the second element 14 does not extend into the lumen 25 of the first element 12. Alternately, the second element 14 does not include a lumen 25. For example, both the first element 12 and the second element 14 may be substantially flat, or gently curved. The elements 12, 14 may be adjacent to one another, or spaced apart from one another, along at least part of their length, particularly where neither element 12, 14 includes a lumen 25. At least one of the elements 12, 14 may be configured to move, such as by sliding, relative to at least part of the other element 12, 14. Alternately, at least part of the second element 14 includes a lumen 25 therein, and a portion of the first element 12 may extend into that lumen 25.

The first element 12 and the second element 14 may both be parts of an integral whole, shaped to constitute the butterfly member 10. For example, the butterfly member 10 may be stamped from a sheet of metal, such as stainless steel. The butterfly member 10 may then be folded, where the first element 12 is on one side of the fold and the second element 14 is on the other side of the fold. At least a portion of each member 12, 14 may be folded into a semicircular or other shape as viewed longitudinally, before or after the folding. Each butterfly member 10 may be fabricated from any suitable material. As one example, at least one butterfly member 10 may be fabricated from any material, such as nickel-titanium alloy, that is elastically or superelastically deformable between the first configuration and the second configuration. As another example, at least one butterfly member 10 may be fabricated from any material, such as stainless steel or plastic, that is plastically deformable between the first configuration and the second configuration. At least part of at least one butterfly member 10 may be plastically deformable between the collapsed configuration and the expanded configuration. At least part of the butterfly member 10 may be annealed, such that it can be plastically deformed without fracturing. Both of the elements 12, 14 may be substantially rigid, such that they are capable of transmitting both compressive and tensile force. Alternately, at least one butterfly member 10 may be configured in a different manner. Alternately, at least one butterfly member 10 may be omitted, and any other suitable structure, mechanism and/or method may be used to register the end effector 4 to tissue.

End Effector

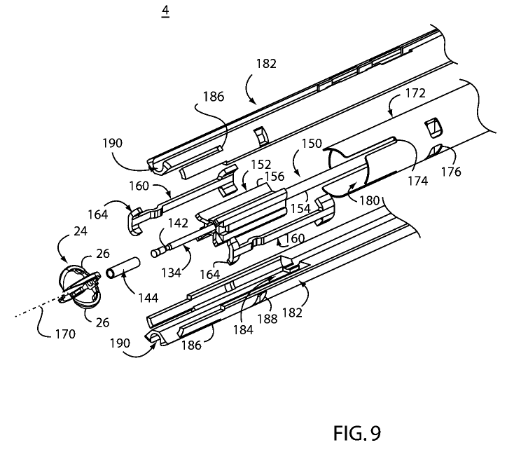

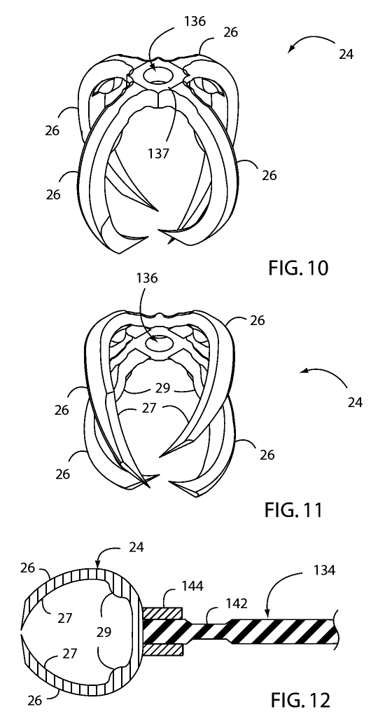

Referring also to FIG. 9, an exploded view of an exemplary end effector 4 is shown. Referring also to FIGS. 10-12, a staple 24 is shown, in a first, initial position. The staple 24 is fixed to a shuttle 134. However, the staple 24 may have a different number of tines, if desired. The staple 24 may be sized and shaped in any suitable manner. The staple 24 may have four tines 26, each extending at least partially in the distal direction. As one example, those four tines 26 may be generally arranged in an X-shape as viewed on end. Alternately, the staple 24 may have more or fewer tines 26. The tines 26 may be curved, and may each have a shape and radius of curvature such that the tines 26 are generally not parallel to one another. The radius of curvature may be substantially coincident with the path of travel of the tines 26 during closure of the staple 24. The staple 24 may be substantially bilaterally symmetrical, although it may be asymmetrical if desired. The distal end of each tine 26 of the staple 24 may have a single substantially pointed or sharpened distal end. However, the distal ends of the tines 26 need not be pointed or sharpened, particularly if the cross-sectional area of each tine 26 is small. Further, the distal end of at least one tine 26 may be bifurcated or split. The distal ends of the tines 26 may be shaped or otherwise configured such that the tines 26 swipe past one another as the staple 24 moves to the closed configuration, as described in greater detail below.

Each tine 26 of the staple 24 may extend proximally from the distal end thereof, outward from the longitudinal centerline of the staple 24, then toward the longitudinal centerline of the staple 24. At its most proximal point, each tine 26 may be oriented generally perpendicular to the longitudinal centerline of the staple 24. However, at least one tine 26 may be oriented differently at its most proximal point. Moving proximally, as each tine 26 approaches the longitudinal centerline of the staple 24, that tine 26 connects to the base 137 of the staple 24. Alternately, each tine 26 may connect directly to the proximal end of at least one other tine 26. Alternately, at least one tine 26 may be curved, shaped and/or oriented in a different manner; the tines 26 need not be shaped in the same manner as one another. Advantageously, the staple 24 is fabricated integrally as a single part, and the tines 26 are individual portions of that single part. As another example, at least one tine 26 may be fabricated separately and later connected to one or more other tines 26 to form the staple 24. Optionally, a splay bump 29 may be positioned on an inner surface 27 of at least one tine 26. The splay bump 29 facilitates splaying of the tine 26, as described in greater detail below.

The staple 24 may be plastically deformable. If so, the staple 24 may be fabricated from stainless steel, titanium or any other suitable plastically-deformable material. Alternately, the staple 24 may be elastically deformable. If so, the staple 24 may be fabricated from nickel-titanium alloy or any other suitable elastic or superelastic material. The staple 24 may be fabricated from a single wire or other piece of material that has a rectangular, circular or other cross-section. The cross-sections of the tines 26 of the staple 24 may be substantially constant along the entire staple 24, or may vary at different locations along the staple 24. For example, the cross-sectional area of the tines 26 of the staple 24 at certain locations may be less than at other locations, in order to promote bending in those locations having a lesser cross-sectional area. An aperture 136 may extend through the base 137 of the staple 24. The aperture 136 may be located coincident with the longitudinal centerline of the staple 24, or may be located at a different position on the staple 24.

A shuttle 134 may be received into and/or through that aperture 136. Advantageously, the shuttle 134 does not extend substantially distally beyond the base 137 of the staple 24. The shuttle 134 may be an elongated rod or generally rod-like structure. At least part of said shuttle 134 may be flexible, such that said shuttle 134 can extend proximally out of the end effector 4 through the tool shaft 6 to the handle 8. Alternately, the shuttle 134 need not extend proximally out of the end effector 4; rather, a cable, rod or other force transmission mechanism may extend along the tool shaft 6 from the shuttle 134 to the handle 8. The distal end of the shuttle 134 is fixed to the staple 24. Alternately, a different or additional part of the shuttle 134 is fixed to the staple 24. The shuttle 134 may be fixed to the staple 24 by welding, by adhesive, by friction fitting, and/or by any other or additional suitable structure, mechanism or method. Alternately, the shuttle 134 and the staple 24 may be fabricated as a single integral structure. The longitudinal centerline of the shuttle 134, at least in proximity to the staple 24, may be substantially coincident with the longitudinal centerline of the staple 24. The shuttle 134 may include a weakened area 142 defined therein, proximal to the staple 24. Advantageously, the weakened area 142 is spaced apart from, but located in proximity to, the staple 24. Alternately, the weakened area 142 may be positioned at a different location on the shuttle 134. The weakened area 142 may be a length of the shuttle 134 having a reduced cross-sectional area compared to the adjacent portions of the shuttle 134. Alternately, the weakened area 142 may be configured in any other manner that provides for separation of the portion of the shuttle 134 distal to the weakened area 142 from the portion of the shuttle 134 proximal to the weakened area 142. As one example, the shuttle 134 may be fabricated as two separate, independent sections, connected at the weakened area 142, and separable at that weakened area 142. Each such portion of the shuttle 134 may include a connector, such that the connectors hold the portions of the shuttle 134 together until separation of the distal portion of the shuttle 134 is desired.

A splay tube 144 may be positioned proximal to the staple 24. Advantageously, the splay tube 144 is positioned in contact with the proximal surface of the base of the staple 24. The splay tube 144 may be fixed to the staple 24, may be connected directly to and separable from the staple 24, or may be separate from the staple 24, whether abutting or spaced apart from the staple 24. The splay tube 144 may be substantially tubular. If so, the distal end of the shuttle 134 is received through the lumen of the splay tube 144, such that the longitudinal centerline of the splay tube 144 is substantially coincident with the longitudinal centerline of the shuttle 134. The splay tube 144 may extend proximally to a location distal to the weakened area 142 of the shuttle 134. Alternately, the splay tube 144 may extend into the weakened area 142 of the shuttle 134, or through the weakened area to a location proximal to the weakened area 142 of the shuttle 134. The splay tube 144 may be generally rigid. Alternately, at least part of the splay tube 144 may be flexible. Alternately, rather than being a separate component, the splay tube 144 may be a part of the shuttle 134 that has a greater cross-sectional area than an adjacent portion of the shuttle 134. The splay tube 144 concentrates bending of the tines 26 of the staple 24 during splaying, as described in greater detail below.

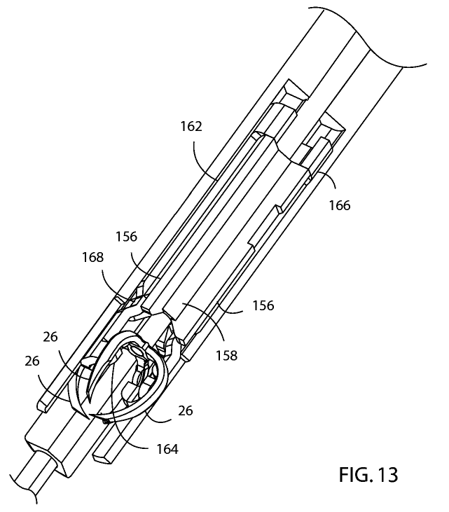

Referring to FIGS. 9 and 13, a driver assembly 150 is located proximal to the staple 24. The driver assembly 150 includes a driver 152 and a driver tube 154 extending proximally from the driver 152. The driver 152 includes a passage defined therethrough, substantially coaxial with the lumen of the driver tube 154, such that a passage extends along the driver assembly 150. The shuttle 134 may extend through that passage in the driver assembly 150, such that the shuttle 134 and the driver assembly 150 are slidable relative to one another, where the driver 152 is slidable along the surface of the shuttle 134. Alternately, the shuttle 134 may be slidable or otherwise movable relative to the driver assembly 150 in another manner, such that at least part of the passage in the driver assembly 150 may be omitted.

The driver 152 may include at least one fin 156 extending radially outward from a centerpiece 158. Advantageously, the driver 152 is fabricated as a unitary assembly. However, at least one fin 156 and the centerpiece 158 may be fabricated separately and assembled together at a later time. The centerpiece 158 is generally elongated and is oriented generally longitudinally, and may have a square, polygonal, circular, or other suitable cross-section. The passage in the driver 152 that receives the shuttle 134 may extend generally longitudinally through the centerpiece 158. Each fin 156 may be generally elongated in the longitudinal direction, and may extend generally radially outward from the centerpiece 158. That is, each fin 156 may lie substantially in a plane, where the longitudinal centerline of the centerpiece 158 lies substantially in that plane. Alternately, at least one fin 156 may extend differently from and/or be oriented differently relative to the centerpiece 158. Each fin 156 may extend outward from any suitable position on the centerpiece 158. Each fin 156 may be oriented to be proximal to and radially aligned with a corresponding tine 26 of the staple 24, such that relative motion of the driver 152 and the staple 24 causes deformation of the tines 26. The distal end of each fin 156 may be shaped to facilitate such deformation in a desired direction or directions, as described in greater detail below. As one example, at least part of the distal end of at least one fin 156 may extend distal to the centerpiece 158. The most radially inward part of the distal end each fin 156 is positioned against the centerpiece 158, and moving outward, the distal end of at least one fin 156 may extend in the distal direction. Moving further outward, the distal end of at least one fin 156 may extend laterally or proximally. The fins 156 may be positioned relative to the centerpiece 158 such that the proximal end of the splay tube 144 abuts the distal end of the centerpiece 158 when the end effector 4 is in an initial configuration. Alternately, the fins 156 may be positioned and/or shaped such that the distal end of at least one fin 156 abuts the splay tube 144.

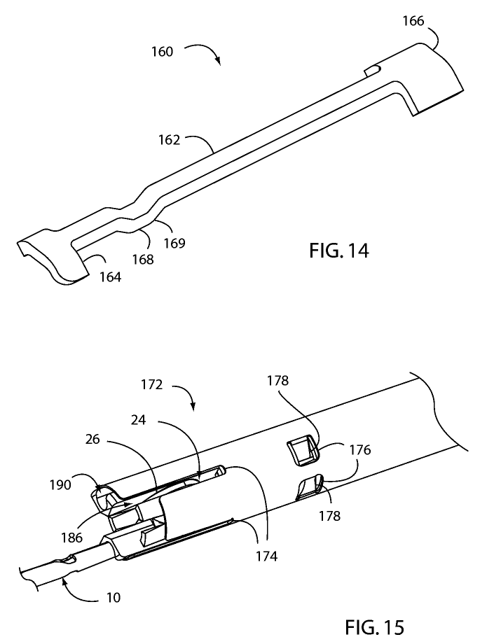

Referring to FIGS. 9 and 13-14, the end effector 4 may include at least one splay arm 160. Each splay arm 160 may include a generally longitudinally oriented strut 162, a distal crossbar 164 connected to the distal end of the strut 162 and generally oriented perpendicular to the strut 162, and a proximal crossbar 166 connected to the proximal end of the strut 162 and generally oriented perpendicular to the strut 162. Advantageously, each splay arm 160 is fabricated as a unitary assembly. However, the strut 162 and/or at least one crossbar 162, 164 may be fabricated separately and assembled together at a later time. The strut 162 may have a generally rectangular cross-section. Alternately, the strut 162 may have any other suitable cross-section along part or all of its length. The strut 162 may be sized and shaped to fit between and/or slide between adjacent fins 156 of the driver 152. Alternately, the strut 162 may be placed differently and/or movable differently relative to the driver 152. The strut 162 may include a trough 168 at or near its distal end, where that trough 168 dips toward the radial centerline of the end effector 4. Alternately, the trough 168 may be positioned or shaped differently, or omitted. When the end effector 4 is in an initial configuration, the trough 168 may be located at or near the distal end of the driver 152, aligned radially between adjacent fins 156 of the driver 152. Alternately, when the end effector 4 is in an initial configuration, the trough 168 may be positioned differently relative to the driver 152.

The distal crossbar 164 may extend generally perpendicularly from the distal end of the strut 162. The distal crossbar 164 also may be curved or angled relative to the strut 162, such that the distal crossbar 164 is positioned at generally the same distance from the longitudinal centerline 170 of the end effector 4 across its length. The distal crossbar 164 may be smoothly curved as a generally arcuate shape, may be incrementally bent as a polygonal approximation of an arcuate shape, or otherwise shaped. Alternately, the distal crossbar 164 may be positioned differently relative to the strut 162 and/or shaped differently. When the end effector 4 is in an initial configuration, each lateral end of the distal crossbar 164 may be positioned between at least one corresponding tine 26 of the staple 24 and the longitudinal centerline 170 of the end effector 4. This positioning facilitates splaying of the tines 26, as described in greater detail below. Alternately, the distal crossbar 164 may be positioned and/or oriented differently relative to the staple 24 when the end effector 4 is in an initial configuration. The proximal crossbar 166 may extend generally perpendicularly from the proximal end of the strut 162. The proximal crossbar 166 also may be curved or shaped relative to the strut 162, such that the proximal crossbar 166 is positioned at generally the same distance from the longitudinal centerline 170 of the end effector 4 across its length. The proximal crossbar 166 may be smoothly curved as a generally arcuate shape, may be incrementally bent as a polygonal approximation of an arcuate shape, or otherwise shaped. Alternately, the proximal crossbar 166 may be positioned differently relative to the strut 162 and/or shaped differently. When the end effector 4 is in an initial configuration, each lateral end of the proximal crossbar 166 may be positioned proximal to the driver 152. Alternately, the proximal crossbar 166 may be positioned and/or oriented differently relative to the driver 152 when the end effector 4 is in an initial configuration.

Referring to FIGS. 9, 13 and 15, the end effector 4 may include a shaft 182. The shaft 182 is shown in FIG. 9 as two separate pieces for clarity, but the shaft 182 need not be fabricated as two or more individual pieces that are later connected; rather, the shaft 182 may be fabricated as a single, unitary piece. The shaft 182 may be a generally cylindrical body having a space 184 defined therein. Alternately, the shaft 182 may be configured in any other suitable manner. As one example, the wall thickness of the shaft 182 may be any suitable amount. As another example, the shaft 182 may be generally tubular. As another example, the shaft 182 need not be tubular in whole or in part, and instead may have any other cross-section in whole or in part that includes a space 184 or other passage therein for receiving one or more other components of the end effector 4. The shaft 182 may include one or more slots 186 defined generally longitudinally therethrough, extending from the distal edge of the shaft 182 in the proximal direction. Each slot 186 is at least as wide as a tine 26 of the staple 24. Alternately, the slots 186 may be positioned and/or oriented differently, or may be omitted altogether. The slots 186 are oriented in radial alignment with the tines 26 of the staple 24, such that each tine 26 is adjacent to and positioned inward from a corresponding slot 186 in the shaft 182. In this way, at least one tine 26 can splay outward through a corresponding slot 186, as described in greater detail below.

The space 184 in the shaft 182 also is advantageously configured to receive the shuttle 134. The shuttle 134 may extend through the tool shaft 6 to the handle 8. Alternately, a cable, wire, rod or other mechanism or structure may extend distally from the handle, along the tool shaft 6, and connect to the shuttle 134 at a location in the end effector 4 or the tool shaft 6. The shaft 182 may extend through the tool shaft 6 to the handle 8. If so, the shuttle 134 may be positioned within the space 184 in the shaft 182 along the length of the tool shaft 6. Alternately, a cable, wire, rod or other mechanism or structure may extend along the tool shaft 6 and connect to the shaft 182 at a location in the end effector 4 or the tool shaft 6. If so, the shuttle 134 may extend out of the proximal end of the shaft 182 and continue to the handle 8 or to a connection between that shuttle 134 and a cable, wire, rod or other mechanism or structure extending distally from the handle 8.

Referring to FIGS. 9, 13 and 15, the end effector 4 may include a tip sleeve 172. The tip sleeve 172 may be a generally cylindrical thin-walled tube. Alternately, the tip sleeve 172 may be configured in any other suitable manner. As one example, the wall thickness of the tip sleeve 172 may be any suitable amount. As another example, the tip sleeve 172 need not be tubular in whole or in part, and instead may have any other cross-section in whole or in part that includes a passage therein for receiving one or more other components of the end effector 4. The tip sleeve 172 is configured to receive the shaft 182 therein. The shaft 182 and/or tip sleeve 172 may be slidable relative to one another, or may be substantially fixed to one another. The tip sleeve 172 may include one or more slots 174 defined generally longitudinally therethrough, extending from the distal edge of the tip sleeve 172 in the proximal direction. Each slot 174 is at least as wide as a tine 26 of the staple 24. Alternately, the slots 174 may be positioned and/or oriented differently, or may be omitted altogether. The slots 174 are oriented in radial alignment with the tines 26 of the staple 24, such that each tine 26 is adjacent to and positioned inward from a corresponding slot 174 in the tip sleeve 172. In this way, at least one tine 26 can splay outward through a corresponding slot 186, as described in greater detail below. One or more tip sleeve windows 176 may be defined through the tip sleeve 172, at a location proximal to the proximal end of one or more of the slots 174. Alternately, one or more tip sleeve windows 176 may be located at a different location on the tip sleeve 172. A tab 178 may extend into at least one tip sleeve window 176. The tab 178 is connected to an edge of a corresponding tip sleeve window 176, extends into that window, and is oriented at least partially toward the longitudinal centerline of the tab sleeve 172. Alternately, at least one tab 178 is configured differently. Each tab 178 may be flexible or rigid. Each tab 178 may be capable of motion relative to a remainder of the tip sleeve 172, or may be substantially immovable relative to a remainder of the tip sleeve 172. The tab 178 may be fabricated by laser-cutting or otherwise cutting through the wall of the tip sleeve 172 along three sides of the tip sleeve window 176, thereby creating a tab 176, which then may be bent toward the longitudinal centerline of the tab sleeve 172.

The tip sleeve 172 includes a lumen 180 or other hollow passage defined completely therethrough in the longitudinal direction. Where the tip sleeve 172 is generally tubular, the lumen 180 is generally cylindrical. However, the lumen 180 may have any suitable shape. The shaft 182, shuttle 134, driver 182, staple 24 and splay arms 164 may be received within the lumen 180 of the tip sleeve 172. Alternately, at least one of those structures is positioned differently within the tip sleeve 172, or is positioned at least partially distal to the end of the tip sleeve 172 or partially proximal to the proximal end of the tip sleeve 172, rather than within it. The slots 174 of the tip sleeve 172 may be aligned with the slots 186 in the shaft 182 to allow splaying of the tines 26 of the staple 24 through the slots 174, 186, as described in greater detail below. The tabs 178 of the tip sleeve 172 may be sized and positioned to fit into the shaft windows 188. The shaft 182 may include one or more troughs 190 defined therein, wherein each trough 190 is sized and shaped to receive a butterfly member 10 therein. Each trough 190 may be generally U-shaped or open, with the open end facing the inner surface of the tip sleeve 172, such that the butterfly member 10 is held between the trough 190 and the tip sleeve 172. Alternately, at least one trough 190 describes a closed shape, or a more-closed shape, such that the corresponding butterfly member 10 is held completely by the shaft 182 alone. Alternately, at least one butterfly member 10 may be connected to the end effector 4 in a different manner. Alternately, at least one butterfly member 10 may be independent of the end effector 4.

Referring also to FIG. 1, the tool shaft 6 extends proximally from the end effector 4. The tool shaft 6 may be flexible or rigid. The tool shaft 6 may be articulated in at least one location, if desired. Referring also to FIG. 2, the tool shaft 6 and the end effector 4 are both sized to pass through a standard sheath 48 used in a catheterization procedure. Optionally, the tool shaft 6 may include a cutaway, trough or other feature (not shown) to allow a guidewire (if any) used in the catheterization procedure to remain in place during actuation of the closure system 2. Alternately, the closure system 2 may include or be configured to follow a second guidewire separate from the one utilized to perform a medical procedure.

The handle 8 is connected to the tool shaft 6, such as to the proximal end of the tool shaft 6. The tool shaft 6 may be fabricated such that the handle 8 is simply the proximal end of the tool shaft 6. Alternately, the tool shaft 6 and the handle 8 may be two separate items that are connected together in any suitable manner. The handle 8 may include any mechanism, mechanisms, structure or structures configured to actuate the end effector 4. For example, as described later in this document, the handle 8 may be configured to actuate the butterfly members 10, shuttle 134 and/or driver assembly 150. Thus, any suitable mechanism or mechanisms that are configured to actuate the butterfly members 10, shuttle 134 and/or driver assembly 150 may be used, as described above. A wire, cable, rod and/or any other suitable structure may extend from the handle 8 through the tool shaft to the butterfly members 10, shuttle 134 and/or driver assembly 150. Alternately, at least one of the butterfly members 10, shuttle 134 and/or driver assembly 150 extends through the tool shaft 6 to the handle 8.

The handle 8 may also include a source of stored energy for actuating the end effector 4. The source of stored energy may be mechanical (such as a spring), electrical (such as a battery), pneumatic (such as a cylinder of pressurized gas) or any other suitable source of stored energy. The source of stored energy, its regulation, and its use in actuating the end effector 4 may be as described in U.S. patent application Ser. No. 11/054,265, filed on Feb. 9, 2005, which is herein incorporated by reference in its entirety. The handle 8 may instead, or also, include a connector or connectors suitable for receiving stored energy from an external source, such as a hose connected to a hospital utility source of pressurized gas or of vacuum, or an electrical cord connectable to a power source.

Alternately, the closure system 2 may include at least two separate components: a butterfly deployment tool connected to and configured to place the butterfly members 10, and a staple placement tool which is connected to the end effector 4 and configured to place the staple 24. In this embodiment, the closure system 2 includes two or more separate tools, in contrast to the closure system 2 disclosed above that is a single integrated tool. The staple placement tool may be slidable relative to the butterfly deployment tool, or vice versa. As one example, at least a portion of the butterfly deployment tool may be tubular, and at least a portion of the staple placement tool may be configured to slide within the lumen of the tubular portion of the butterfly deployment tool. As another example, the butterfly deployment tool and/or the staple placement tool may include a groove defined therein, where the other tool includes a rail, rib or other structure configured to slide along that groove.

Operation

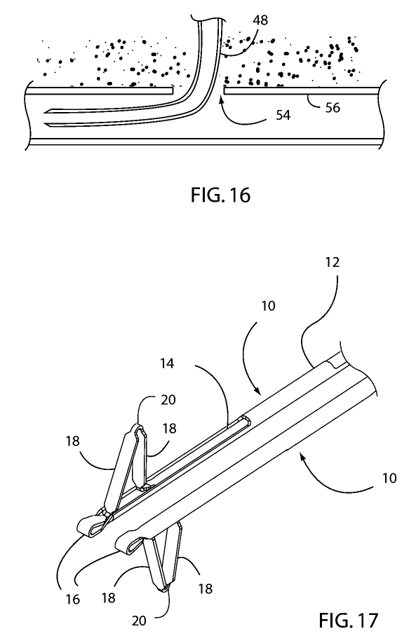

Referring to FIGS. 7 and 16, in the course of a standard catheterization procedure, a sheath 48 is inserted through a passage 53 in tissue 52 such that one end of the sheath 48 enters an opening 54 in a blood vessel 56. The passage 53 extends between the epidermis 55 of the patient and the opening 54 in the blood vessel 56. The sheath 48 may be advanced any suitable distance into the blood vessel 56, as determined by the physician performing the procedure. As an example of a catheterization procedure, the blood vessel 56 may be a femoral artery, and the tissue 52 may be the tissue of the leg between the surface of the leg and the femoral artery. However, the blood vessel 56 may be a different blood vessel, and the tissue 52 may be different tissue in the vicinity of that different blood vessel. As one example, the blood vessel 56 may be the radial artery or the carotid artery. During the catheterization procedure, any suitable tools are utilized to perform the desired treatment on the patient, such as the placement of one or more stents in the coronary arteries or peripheral vessels of the patient. After the treatment has been performed, the tools utilized to perform that treatment are removed from the patient via the sheath 48, and the sheath 48 is left in place.

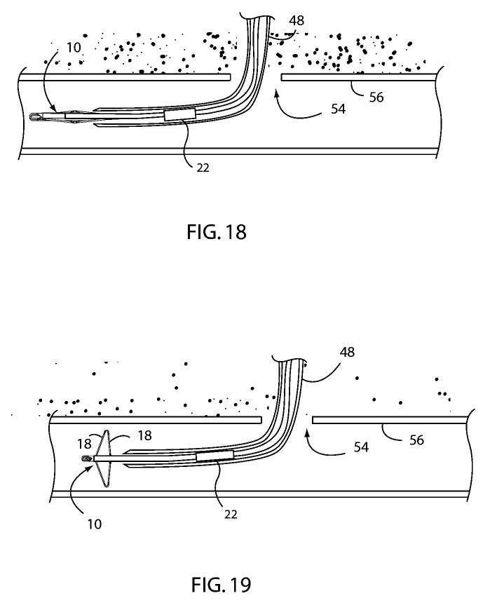

Referring also to FIG. 18, the end effector 4 of the closure system 2 is inserted into the sheath 48. The end effector 4 may be advanced along the sheath 48 in any suitable manner. As one example, the end effector 4 is manually pushed along the sheath 48 by the physician or other user by applying a force to the tool shaft 6 and/or the handle 8 after the end effector 4 has entered the sheath 48. Each butterfly member 10 initially may be in its first, collapsed configuration as the end effector 4 is advanced along the sheath 48. The end effector 4 continues to advance distally into the sheath 48 until at least the distal end 16 of at least one butterfly member 10 is distal to the distal end of the sheath 48. That is, the end effector 4 is advanced along the sheath 48 until at least the distal end 16 of at least one butterfly member 10 is outside of the lumen of the sheath 48. This position of the end effector 4 relative to the sheath 48 may be referred to as the standby position. The sheath 48 has a known length, and at least part of the end effector 4 is advanced along the lumen of the sheath 48 a distance greater than the length of the sheath 48. Thus, the particular position of the distal end of the sheath 48 in the lumen of the blood vessel 56 need not be known in order for the end effector 4 to be advanced to the standby position. Optionally, one or more markings may be placed on the tool shaft 6, such that when those one or more markings enter the lumen of the sheath 48, the end effector 4 has been advanced to the standby position. The marking or markings on the tool shaft 6 are placed at a distance from the distal end of the end effector 4 that is greater than the length of the sheath 48. The distal ends of the butterfly members 10 may be cushioned, as described above, such as by the use of a spring 130, soft tip 19, or other suitable feature or features. As a result, as the butterfly members 10 advance distal to the distal end of the sheath 48 into the lumen of the blood vessel 56, the back wall of that blood vessel 56 opposite the opening 54, as well as other tissue of the blood vessel 56, is protected.

Optionally, a guidewire (not shown) utilized in the catheterization procedure may remain in the lumen of the sheath 48, and the end effector 4 and tool shaft 6 may follow that guidewire in any suitable manner. As one example, where a cutaway, groove or other feature is defined in the end effector 4 and/or tool shaft 6, that feature may slide along the guidewire. Optionally, the guidewire used in the catheterization procedure is removed from the lumen of the sheath 48 prior to the introduction of the end effector 4 into the sheath 48, and a second, thinner guidewire configured for use with the closure system 2 is inserted through the lumen of the sheath 48 and into the lumen of the blood vessel 56. The original guidewire may be removed before or after the placement of the second guidewire. The second, thinner guidewire, if used, may be more convenient to remove from the opening 54 in the blood vessel 56 after the staple 24 has been closed.

After the end effector 4 is in the standby position, at least one butterfly member 10 is actuated to move from its first, collapsed configuration to its second, expanded configuration. This actuation may be performed in any suitable manner. Referring also to FIG. 17, as one example, the second element 14 of each butterfly member 10 is held substantially in place, and the first element 12 of each butterfly member 10 is pulled proximally in any suitable manner. As one example, the elements 12, 14 each extend through the tool shaft 6 to the handle 8, and a mechanism or mechanisms in the handle 8 push or otherwise move the first element 12 proximally. As another example, the first element 12 is connected to a cable or other force transmission member, and the handle 8 exerts a proximal force on that cable, which in turn moves the first element 12 proximally.

Proximal motion of the first element 12 relative to the second element 14 exerts a compressive force on the segments 18, substantially in the longitudinal direction. Because at least one segment 18 is angled, curved or otherwise offset from the longitudinal direction, that longitudinal force results in a moment that acts on at least part of at least one segment 18. As a result of that moment, each segment 18 rotates outward from the longitudinal centerline of the first element 12 about the hinge member 20 as well as about the point of connection between each segment 18 and a remainder of the first element 12. The hinge member 20 allows the segments 18 to rotate relative to one another at a defined point, by providing a weakened area or other feature that is configured to bend upon the application of a force that is less than the amount of force needed to bend the segments 18 themselves. The deflection of the segments 18 as a result of the application of moments thereto may be plastic deformation. Alternately, that deflection may be elastic deformation. After the segments 18 of a butterfly member 10 complete their deflection, that butterfly member 10 is in the second, expanded configuration, as shown in FIG. 19. The sheath 48 may then be removed. Alternately, the sheath 48 may be removed before one or more butterfly members 10 expand to the second configuration. As another example of a butterfly member 10, the segments 18 may be bendable, rather than deformable. Alternately, a single segment 18, rather than two separate segments, is provided. As another example, the first element 12 of each butterfly member 10 is held substantially in place, and the second element 14 of each butterfly member 10 is pushed distally, such as by a rod or other rigid linkage attached to the end of each second element 14. This motion of the second element 14 relative to the first element 12 exerts a compressive force on the segments 18, which then deform to the second, expanded configuration substantially as described above.

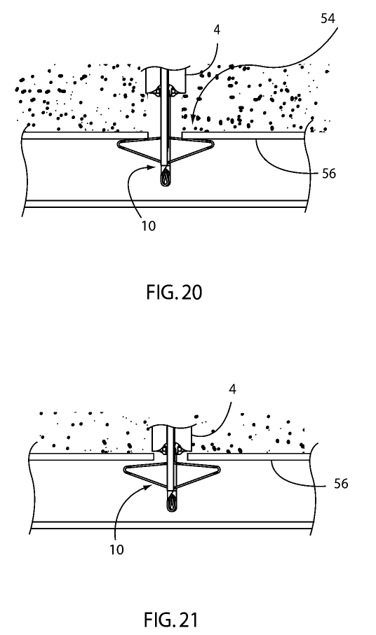

Next, referring also to FIG. 20, the closure system 2 is moved proximally until the expanded butterfly member or members 10 contact the inner wall of the blood vessel 56, in proximity to the opening 54. The butterfly members 10 are held substantially stationary relative to the housing 22 as the closure system 2 is moved proximally. The closure system 2 may be moved proximally in any suitable manner. As one example, the handle 8 is manually moved proximally, causing the expanded butterfly member or members 10 to contact the inner wall of the blood vessel 56.

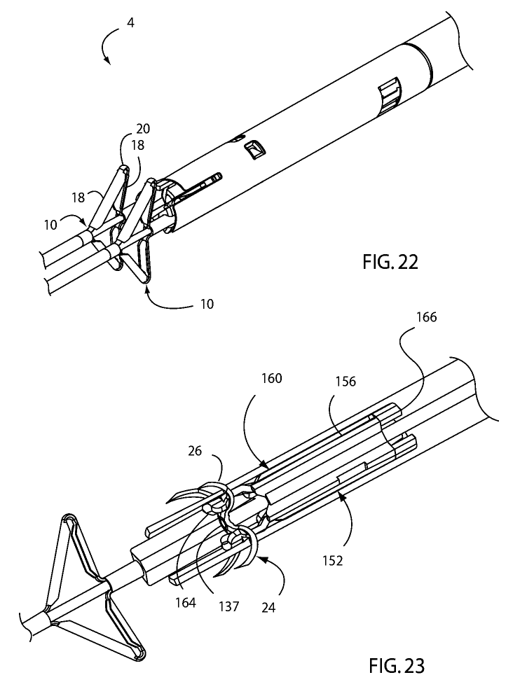

Referring also to FIGS. 21-22, each butterfly member 10 then may be moved proximally while the remainder of the end effector 4 is held in a substantially constant position. The butterfly members 10 are moved such that each butterfly member 10 is maintained in an expanded configuration as it moves proximally. As a result, the expanded portion of each butterfly member 10 pulls the wall of the blood vessel 56 toward the distal end of the tip sleeve 172, capturing the wall of the blood vessel 56 and registering the opening 54 in the blood vessel 56 to the distal end of the tip sleeve 172. The expanded portion of each butterfly member 10 may be wider than the opening 54 to facilitate this motion of the wall of the blood vessel 56. Advantageously, the expanded portion of each butterfly member 10 may be moved within one-half millimeter of the distal end of the tip sleeve 172. However, the distance that the expanded portion of each butterfly member 10 is moved may be more or less. Alternately, at least one butterfly member 10 is moved relative to a force, rather than a distance. That is, a particular force is exerted proximally on the butterfly member 10, causing it to move proximally until the force exerted on the butterfly member 10 by the wall of the blood vessel 56 in the distal direction is substantially equal to the force exerted on the butterfly member 10 in the proximal direction. Thus, the wall of the blood vessel 56 is moved into position in preparation for stapling. The wall of the blood vessel 56 is held in position against the distal end of the tip sleeve 172 by compressive force exerted against the tip sleeve 172 by the expanded portion of each butterfly member 10. The movement of each butterfly member 10 may be accomplished in any suitable manner. For example, at least one element 12, 14 of at least one butterfly member 10 extends to the handle 8, and at least one of those elements 12, 14 is actuated directly by a mechanism or mechanisms associated with the handle 8. As another example, both the first and the second elements 12, 14 of at least one butterfly member 10 may be moved proximally by a cable or cables attached to the elements 12, 14. Alternately, the expanded portion of each butterfly member 10 is held substantially stationary, and the remainder of the end effector 4 is advanced distally. Such motion of the remainder of the end effector 4 may be accomplished in a manner similar to that described above with regard to the motion of the butterfly members 10. For example, each butterfly member 10 may be held substantially stationary relative to the handle 8, which in turn is held substantially stationary relative to the blood vessel 56. A force in the proximal direction is then exerted on the tip sleeve 172, such as via a member capable of transmitting compressive force, where that member extends through the tool shaft 6 to the handle 8.

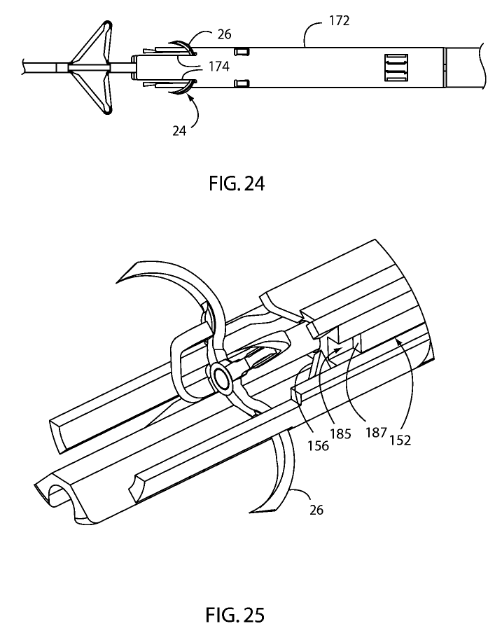

Next, referring also to FIGS. 23-24, at least one splay arm 160 may retract proximally while the staple 24 may be held substantially in place. The splay arm or arms 160 may be retracted in any suitable manner. As one example, the driver 152 may be retracted proximally in any suitable manner; as one example, the driver 152 may be connected to the handle 8 via a cable (not shown) that is retracted proximally by a mechanism or mechanisms in the handle 8. As the driver 152 moves proximally, the proximal end of at least one fin 156 contacts the proximal crossbar 166 of at least one corresponding splay arm 160. Further proximal motion of the driver 152 causes the proximal end of at least one fin 156 to urge the corresponding at least one splay arm 160 in the proximal direction. Alternately, at least one splay arm 160 may be moved proximally in any other suitable manner, such as by the use of a cable or other force transmission member (not shown) that is connected to at least one splay arm 160 and that may extend through the tool shaft 6 to the handle 8, such that application of tension and/or proximal force to that cable or other force transmission member causes proximal motion of the corresponding at least one splay arm 160.

During proximal motion of the splay arm 160, the staple 24 may be held substantially in place, such as by holding the shuttle 134 in place. The staple 24 may be fixed to the shuttle, as described above, such that holding the shuttle 134 in place holds the staple 24 in place. Optionally, the weakened area 142 in the shuttle may be configured to be stronger in compression than in tension. As each splay arm 160 moves proximally, the distal crossbar 164 of that splay arm 160 moves proximally as well. Each distal crossbar 164 may be positioned distal to a splay bump 29 on a corresponding tine 26 of the staple 24. Alternately, if a splay bump 29 is not used on a tine 26, the corresponding distal crossbar 164 is positioned distal to at least part of that tine 26.

As the splay arm 160 retracts proximally, the distal crossbar 164 of that splay arm 160 contacts and then exerts a proximal force on the corresponding splay bump 29 of at least one corresponding tine 26 of the staple 24. If the splay bump 29 is omitted, the distal crossbar 164 exerts a proximal force on a different portion of the tine 26 of the staple 24. The shuttle 134 substantially restrains the base 137 of the staple 24 against proximal motion, such that the longitudinal position of the base 137 of the staple 24 remains substantially unchanged as the distal crossbar 164 exerts proximal force on the splay bump 29 of at least one tine 26. However, the tines 26 of the staple 24 are not substantially restrained against motion resulting from application of force to the staple 24 by the distal crossbar 164. The distal crossbar 164 is positioned sufficiently far from the longitudinal centerline of the staple 24 such that the exertion of proximal force by that distal crossbar 164 against a corresponding splay bump 29 (which itself is spaced apart from the longitudinal centerline of the staple 24) generates a moment about the base 137 of the staple 24. This moment causes the corresponding tine 26 of the staple 24 to move outward from the longitudinal centerline of the staple 24. Thus, as the distal crossbar or crossbars 164 each exert a force on the corresponding tine or tines 26, the distal ends of the tines 26 each move in a direction having a component of motion away from the longitudinal centerline of the staple 24. This deformation of the staple 24 may be referred to as “splaying,” as shown in FIG. 23. The splay tube 144 acts to localize bending of the tines 26 of the staple 24 at or in proximity to the junction between the splay tube 144 and the base 137 of the staple 24. That is, the splay tube 144 supports the portion of the base 137 of the staple 24 that is directly in contact with the splay tube 144, thereby reducing or preventing bending of that portion of the base 137. As a result, the splay tube 144 encourages bending of the tines 26 of the staple 24 about a point at least as far from the longitudinal centerline 170 of the end effector 4 as the radius of the splay tube 144 itself. Alternately, the splay tube 144 may be omitted, such that bending of the tines 26 of the staple 24 may be localized at the junction between the shuttle 134 and the base 137 of the staple 24, localized at a different location, or not localized at all. During splaying of the staple 24, the tines 26 themselves may remain substantially undeformed; rather, a portion of the tines 26 distal to the base 137 of the staple 24 may deform. Alternately, at least one tine 26 may deform during splaying of the staple 24. Alternately, the splay arms 160 may be held substantially in place, and the staple 24 may be advanced distally, in order to splay the staple 24. The splay tube 144 may also act to protect the weakened area 142. The weakened area 142 may be located within the lumen of the splay tube 144. If so, the splay tube 144 transmits some or all of the compressive force to the staple 24, protecting the weakened area 142 from the application of force and preventing the weakened area 142 from fracturing. In this way, the splay tube 144 may protect and/or reinforce the weakened area 142.

Where the staple 24 is made from a plastically-deformable material such as stainless steel, the staple 24 deforms plastically as it splays from its initial configuration to the splayed configuration. Plastic deformation is deformation that remains after the load that caused it is removed, or that would remain if the load were removed. Alternately, the staple 24 may be elastically-deformable from its initial configuration to the splayed configuration. The staple 24 may be spring-loaded inwards to the initial configuration, such that the staple 24 springs outward and returns to the splayed configuration upon application of force or upon movement to a position relative to the tip sleeve 172 such that the staple 24 is free to spring outward. Referring to FIG. 24, as the distal ends of the tines 26 move away from the longitudinal centerline of the staple 24, at least part of each tine 26 may move outside the tip sleeve 172 through a slot 174 or other opening in the tip sleeve 172. As a result, the tines 26 of the staple 24 may move apart from one another a distance greater than the diameter of the tip sleeve 172.

After the staple 24 has deformed to a splayed configuration, as shown in FIGS. 23-24, the driver 152 ceases its proximal motion, and then begins to move distally. The driver 152 may idle for any suitable length of time before changing direction, or may change its direction of motion without substantially idling. Referring also to FIG. 25, the driver 152 may include one or more notches 185 defined at or in proximity to its distal end, where each notch 185 may include a wall 186 at its proximal end facing generally in the distal direction. At least one wall 187 may be substantially planar and substantially perpendicular to the longitudinal centerline 170 of the end effector 4. Each notch 185 may be positioned radially between two fins 156 of the driver 152. Each notch 185 is substantially radially aligned with the trough 168 of the corresponding splay arm 160. Thus, as the driver 152 advances distally, each notch wall 187 may engage the corresponding trough 168 of the corresponding splay arm 160. Because the splay arms 160 are restrained against radial motion away from the longitudinal centerline 170 of the end effector 4, engagement between each notch wall 187 and the trough 168 of the corresponding splay arm 160 pushes that splay arm 160 forward as the driver 152 moves distally. As the splay arms 160 move distally, the distal crossbars 164 of the splay arms 160 move distally away from the staple 24.

As the driver 152 continues to move distally, the distal end of at least one fin 156 contacts a corresponding tine 26 of the staple 24. At this time, or at any time between splaying of the staple 24 and contact between the driver 152 and the staple 24, the shuttle 134 is released such that it can travel freely. Consequently, when the driver 152 contacts the staple 24, such contact pushes the staple 24 distally, which in turn moves the shuttle 134 distally. The motion of the staple 24 between its splaying and its later cessation of motion may be referred to as “shuttling.” Referring also to FIG. 15, the driver 152 continues to move distally, the splay arms 160 continue to move distally until the proximal crossbar 166 of each splay arm 160 contacts one or more corresponding tabs 178 that extend at least partially inward from a remainder of the tip sleeve 172. Each proximal crossbar 166 and at least one corresponding tab 178 are oriented relative to one another such that each proximal crossbar 166 contacts at least one corresponding tab 178 during distal travel of the corresponding splay arm 160. Contact between the proximal crossbar 166 of a splay arm 160 and at least one corresponding tab 178 causes that splay arm 160 to cease its distal motion. That is, the tabs 178 of the tip sleeve 172 act as a hard stop to restrain the splay arms 160 against further motion in the distal direction. Alternately, the splay arms 160 may be restrained against further motion in the distal direction in any other suitable manner. As an example, the proximal crossbar 166 of at least one splay arm 160 may contact a feature of the tip sleeve 172 other than the tab 178. As another example, the proximal crossbar 166 of at least one splay arm 160 may contact a feature extending from the shaft 182.

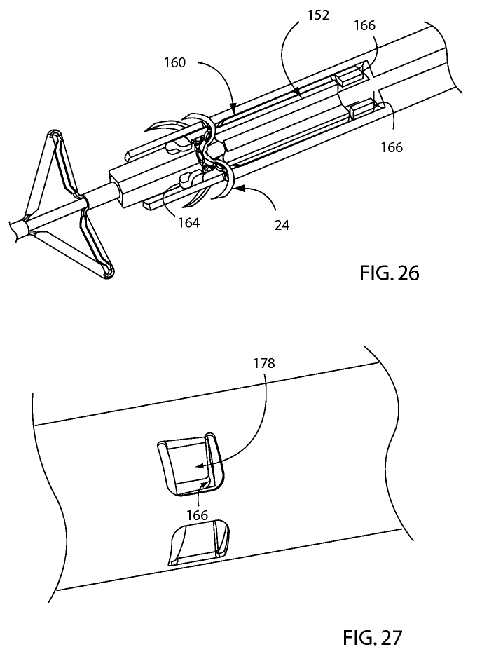

After the splay arms 160 cease their motion in the distal direction, the driver 152 may continue its motion in the distal direction. Referring also to FIGS. 9, 14 and 26, as the driver 152 continues its distal motion, the splay arm 160 is held stationary relative to the moving driver 152 as a result of contact between the proximal crossbar 166 and at least one tab 178. As a result, each notch 185 in the distal end of the driver 152 begins to exert a force in the distal direction on the corresponding trough 168 of the corresponding splay arm 160. More particularly, the wall 187 of each notch 185 may exert a distal force on the corresponding trough 168. The distal end of each splay arm 160 is substantially free. Further, each trough 168 includes a segment 169 that is angled and/or curved partially toward the longitudinal centerline 170 of the end effector 4. Consequently, application of a force in the distal direction on that angled and/or curved segment 169 by the driver 152 results in a component of force on the splay arm 160 in a direction perpendicular to the longitudinal centerline 170 of the end effector 4, causing the distal end of the splay arm 160 to begin to bend outward. The material from which each splay arm 160 is fabricated is selected to allow such bending. Thus, the distal crossbars 164 at the ends of the splay arms 160 begin to spread out from the longitudinal centerline 170 of the end effector 4 and from one another. Additionally, as the driver 152 continues to move distally, it continues to push the staple 24 distally, and thereby continues to push the shuttle 134 distally. As the staple 24 moves distally, the tines 26 begin to penetrate tissue 56 on opposite sides of the opening 54. Alternately, the tines 26 enter tissue 56 at an earlier or later time.

As the driver 152 continues to move distally, it continues to bend the distal ends of the splay arms 160 further away from the longitudinal centerline 170 of the end effector 4, as each notch 185 slides along and thereby pushes out against the corresponding segment 169 of the corresponding splay arm 160. Eventually, the driver 152 moves far enough distally that the innermost portion of the trough 168 of each splay arm 160 rides up over the wall 187 and onto the centerpiece 158 of the driver 152, between adjacent fins 156. The centerpiece 158 may be shaped such that it is substantially flat between each pair of adjacent fins 156, and/or substantially equally spaced from the longitudinal centerline 170 of the end effector 4 along its length. As a result, when the trough 168 of a splay arm 160 rides up over the wall 187 of the corresponding notch 185 and onto the centerpiece 158, the distal ends of the splay arms 160 have spread out from the longitudinal centerline 170 of the end effector 4 a maximum amount. Alternately, the centerpiece 158 and/or the splay arm 160 may be configured to allow for change in the degree of spacing between the distal end of that splay arm 160 and the longitudinal centerline 170 of the end effector 4 as the driver 152 continues to move distally. At this time, the distal ends of the splay arms 160 are spaced away from the longitudinal centerline 170 of the end effector 4 a sufficient distance to allow the splayed tines 26 of the staple 24 to move distal to the distal crossbars 164 of the splay arms 160.

Distal motion of the shuttle 134 is then stopped. As one example, a portion of the shuttle 134 encounters a hard stop in the end effector 4 or handle 8 that prevents the shuttle 134 from moving any further in the distal direction. As another example, the shuttle 134 may be controlled to stop in any other suitable manner. As another example, distal motion of the shuttle 134 is not stopped, but slowed, prior to or instead of stopping the distal motion of the shuttle 134. As another example, motion of the shuttle 134 is reversed, such that the shuttle 134 begins to move in the proximal direction. As another example, distal motion of the shuttle 134 may be stopped before at least one trough 168 of a splay arm 160 rides up and out of the corresponding notch 185 in the driver 152.

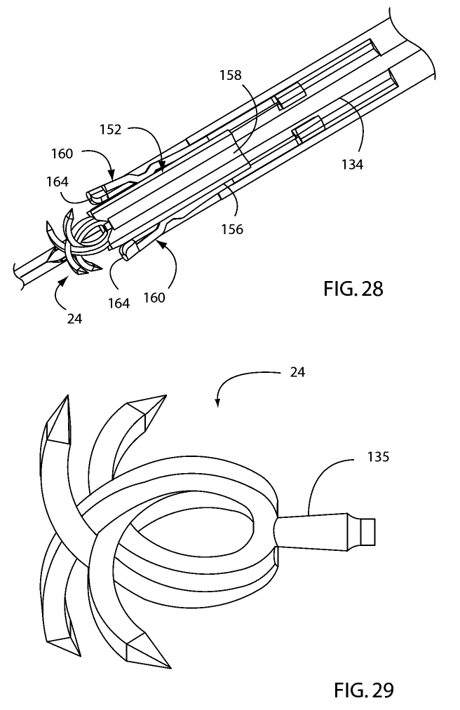

When distal motion of the shuttle 134 has stopped, motion of the staple 24 consequently stops as well. However, the driver 152 continues to move distally, thereby contacting and then applying a force in the distal direction to the staple 24. Referring also to FIGS. 13 and 28, the distal end 153 of each fin 156 may be radially aligned with a corresponding tine 26 of the staple 24. Further, the distal end 153 of each fin 156 may be spaced apart from the longitudinal centerline 170 of the end effector 4. As described above, the staple 24 is fixed to the shuttle 134, such as the aperture 136 that may extend through the base 137 of the staple 24. As a result, the distal end 153 of each fin 156 exerts a distal force on a corresponding tine 26 of the staple 24 at a location on that tine that is offset from the longitudinal centerline 170 of the end effector 4, resulting in a moment about the intersection of the staple 24 and the shuttle 134. Each tine 26 of the staple 24 that experiences that moment moves toward the longitudinal centerline 170 of the end effector 4, which may be substantially the same as the longitudinal centerline 170 of the staple 24. In the course of this motion, the distal ends of the tines 26 may first move toward the longitudinal centerline 170 of the end effector 4 and toward one another, cross each other, then move away from the longitudinal centerline 170 of the end effector 4 and away from one another. The tines 26 need not substantially change shape as they move; rather, they may rotate about the intersection of the staple 24 and the splay tube 144, the staple 24 and the shuttle 134, or any other suitable pivot point. Alternately, one or both of the tines 26 may deform, at least in part, as they move. The radius of curvature of each tine 26 may be substantially coincident with its path of travel during closure of the staple 24. Deformation of the staple 24 as a result of contact between the staple 24 and the driver 152 may be referred to as “closing” the staple 24. This deformation may be plastic deformation from the splayed configuration to a final, closed configuration.

The tines 26 of the staple 24 may be fabricated such that they are out of plane with one another when the staple 24 is in the initial configuration and in the splayed configuration, such that at least two of the tines 26 may swipe past one another and do not substantially interfere with one another during closing of the staple 24. In particular, opposed tines 26 of the staple 24 may be out of plane relative to one another, such that they substantially do not contact one another during closing. As another example, one or more tines 26 come into contact with one another during closing of the staple 24. Such contact may still allow at least two tines 26 to swipe past one another, or alternately may cause at least two tines to lock into place relative to one another or otherwise engage one another. Optionally, the distal ends of the tines 26 of the staple 24 may be shaped substantially conically. As the staple 24 closes, the conical tips of two or more tines 26 may come into contact with one another. If so, the shape of the conical tips results in this contact causing the tines 26 to slide adjacent to one another instead of interfering with one another. Alternately, the distal end of each tine 26 is substantially planar, where each plane is oriented in a different direction. As a result, if the distal ends of the tines 26 encounter one another, contact between the differently-oriented planes at the distal ends of the tines pushes the tines 26 out of plane relative to one another. Alternately, the tines 26 of the staple 24 are plastically deformed out of plane with one another while the staple 24 is splayed open and/or being closed.

When deformation of the tines 26 of the staple 24 is complete, the staple 24 is in the closed configuration. In that closed configuration, at least part of each tine 26 of the staple may be located within the lumen of the blood vessel 56. The tines 26 may be positioned such that a part of each tine 26 is positioned against an inner surface of the blood vessel 56. Alternately, the tines 26 may be positioned differently relative to the wall of the blood vessel 56. In the closed configuration, the staple 24 holds opposite sides of the opening 54 together, substantially closing the opening 54.

After the staple 24 has been closed, it remains connected to the shuttle 134, which is stationary. The driver 152 continues to be urged in the distal direction, but the fins 156 of the driver 152 contact the closed tines 26 of the stationary staple 24, such that the driver 152 can no longer move distally. As a result, the driver 152 exerts a distal force on the staple 24, which results in a tensile force on the shuttle 134 and thereby on the weakened area 142. The weakened area 142 of the shuttle 134 is shaped and sized, or otherwise configured, such that this tensile force is sufficient to fracture the weakened area 142. As one example, the weakened area 142 may have a cross-sectional area sufficiently small such that the tensile force exerted by the driver 152 is sufficient to cause the weakened area 142 to fracture. The driver 152 may be urged distally with a constant force throughout the actuation of the end effector 4, through closing of the staple 24 and fracturing of the weakened area 142. Optionally, the driver 152 may be controlled to apply an increased distal force to the staple 24 after the staple 24 has been closed. If so, the weakened area 142 of the shuttle 134 may be shaped and sized, or otherwise configured, such that the weakened area 142 does not fracture until the increased distal force is applied to it. The distal force applied to the weakened area 142 after the staple 24 has been closed may be greater than the proximal force applied to the weakened area 142 while the staple 24 is being splayed. Alternately, the distal force may be substantially equal in strength (but not direction) to the proximal force, and the weakened area 142 may be configured to be stronger in compression than in tension, such that the weakened area 142 does not fracture in compression but fractures in tension. Because the splay tube 144 is not fixed to the weakened area 142, the splay tube 144 does not reinforce or support the weakened area 142 as tension is applied to the shuttle 134, such that the splay tube 144 does not substantially experience or transmit the tensile force applied to the shuttle 134.

Referring also to FIG. 29, fracture of the weakened area 142 of the shuttle 134 separates the fragment 135 of the shuttle 134 distal to the weakened area 142, and the closed staple 24 which is connected to that fragment 135 of the shuttle 134, from the end effector 4. The closed staple 24 and portion of the shuttle 134 distal to the weakened area 142 are then free to exit the distal end of the end effector 4.

Next, each butterfly member 10 is deformed from the expanded configuration back to the collapsed configuration. This deformation may be performed by reversing the steps described above for deforming the butterfly member 10 from the collapsed configuration to the expanded configuration. Where at least one butterfly member 10 elastically deformed from the collapsed configuration to the expanded configuration, force exerted on that butterfly member 10 to maintain the butterfly member in the expanded configuration is simply released, allowing the butterfly member 10 to return to the collapsed configuration.

After each butterfly member 10 returns to the collapsed position, the end effector 4 is moved proximally, and the butterfly members 10 then exit from the opening 54. As the end effector 4 is moved away from the opening 54, the staple 24 exits the distal end of the tip sleeve 172 and shaft 182, and/or other components of the end effector 4, because the staple 24 grasps the tissue 56 with greater force than any remaining frictional forces or other incidental forces holding it to the end effector 4. The end effector 4 thus passively releases the closed staple 24, because the end effector 4 need not exert a force on the closed staple 24 to eject it. The closed staple 24, and the fragment 135 of the shuttle 134 located distal to the fracture in the weakened area 142, remain in place in the patient. As another example, the closed staple 24 may be actively ejected from the end effector 4, by pushing the closed staple 24 out of the end effector 4 in any suitable manner. The guidewire, if used, is then removed from the blood vessel 56. Alternately, the guidewire may be removed at a different time. The guidewire is pulled out of the blood vessel 56 adjacent to the closed staple 24 and between the edges of what had been the opening 54 in the blood vessel 56. Thus, a smaller-diameter guidewire may be advantageous, as it may leave a smaller gap in tissue between the edges of what had been the opening 54 in the blood vessel, such that the wall of the blood vessel can rebound more quickly to close that gap. After the end effector 4 is removed from the patient, the sheath 48 is removed if it is still present in the patient. The procedure is complete, and the opening 54 is substantially closed.

Where the closure system 2 includes a separate butterfly deployment tool connected to and configured to place the butterfly members 10, and a separate staple placement tool which is connected to the end effector 4 and configured to place the staple 24, each of the two separate components may be substantially as described above with regard to the single, integrated tool, with minor variations. First, the butterfly deployment tool may be inserted through the opening 54 in the blood vessel 56 and actuated such that a part of each butterfly member 10 is in the expanded configuration and seated against the inner surface of the wall of the blood vessel 56. Then, the staple placement tool may be slid along the butterfly deployment tool toward the opening 54 in any suitable manner, and actuated substantially as described above. The staple 24 is closed, and the weakened area 142 fractured to separate the staple 24 and a portion of the shuttle 134 distal to the weakened area 142 from the end effector 4, substantially as described above. The staple placement tool is then withdrawn. After the staple placement tool is withdrawn, the butterfly deployment tool is withdrawn, the opening 56 is substantially closed, and the procedure is complete.

Motion of any of the components of the end effector 4 described above may be controlled in any suitable manner. For example, one or more of those components may extend through the shaft 6 to the handle 8, where the handle 8 directly applies a force to and/or directly controls the motion of each such component. As another example, one or more of those components may be connected to a cable or other force transmission member (not shown) which extends through the shaft 6 to the handle 8, where the handle 8 applies a force to and/or otherwise controls each such cable or other force transmission member. The particular manner of control of the motion of components of the end effector 4 is not critical.

Operation: Closure of Other Tissue Openings

Referring to FIGS. 1-2, the closure system 2 may be used to close any suitable opening in tissue. If so, the operation of the closure system 2 is substantially as described above. As one example, the closure system 2 may be used to close a trocar port or other surgical opening in the body of the patient. As another example, the closure system 2 may be used to close a wound in the body of the patient, whether on the skin of the patient or in the interior of the patient's body. As another example, the closure system 2 may be used to repair a hernia at any suitable location in the patient's body. As another example, the closure system 2 may be used for catheter-based and/or guidewire-based interventions, such as PFO closure, gastrointestinal surgery, bariatric surgery, closure of openings in the stomach made in the course of natural orifice surgery (NOTES), or any other suitable procedure. Where the closure system 2 is used for catheter-based and/or guidewire-based interventions, the shaft 6 may be substantially flexible, in whole or in part, to facilitate its advancement through tissue passages in conjunction with the catheter or and/or guidewire. Depending on the particular procedure for which the staple 24 is utilized, the action of splaying the staple 24 may be omitted.

While the invention has been described in detail, it will be apparent to one skilled in the art that various changes and modifications can be made and equivalents employed, without departing from the present invention. It is to be understood that the invention is not limited to the details of construction, the arrangements of components and/or the details of operation set forth in the above description or illustrated in the drawings. Headings and subheadings are for the convenience of the reader only. They should not and cannot be construed to have any substantive significance, meaning or interpretation, and should not and cannot be deemed to be limiting in any way, or indicate that all of the information relating to any particular topic is to be found under or limited to any particular heading or subheading. The contents of each section of this document are merely exemplary and do not limit the scope of the invention or the interpretation of the claims. Therefore, the invention is not to be restricted or limited except in accordance with the following claims and their legal equivalents.

What is claimed is:

1. A medical device for closing an opening in tissue, comprising:
a staple including a plurality of tines, wherein said staple is deformable from an initial configuration to a splayed configuration and then a closed configuration; and
a shuttle fixed to said staple, wherein said shuttle includes a weakened area proximal to said shuttle.

2. The medical device of claim 1, incorporating by reference all of the limitations of that claim, wherein said shuttle is a rod.

3. The medical device of claim 1, incorporating by reference all of the limitations of that claim, wherein said weakened area is a portion of the length of said shuttle with a smaller cross sectional area than a remainder of said shuttle.

4. The medical device of claim 1, incorporating by reference all of the limitations of that claim, wherein said weakened area is stronger in compression than in tension.

5. The medical device of claim 1, incorporating by reference all of the limitations of that claim, wherein said staple includes a base from which said tines extend, wherein said shuttle is fixed to said base.

6. The medical device of claim 1, incorporating by reference all of the limitations of that claim, further comprising a splay tube positioned around said shuttle proximal to and in contact with said staple.

7. The medical device of claim 6, incorporating by reference all of the limitations of that claim, wherein said splay tube is positioned around said weakened area.

8. A medical device for closing an opening in tissue, comprising:
a staple including a plurality of tines, wherein said staple is deformable from an initial configuration to a splayed configuration and then a closed configuration; and
a shuttle attached to and extending proximally from said staple; and
a driver slidable along the surface of said shuttle.

9. The medical device of claim 8, incorporating by reference all of the limitations of that claim, wherein said staple, said shuttle and said driver are all arranged substantially coaxially.

10. The medical device of claim 8, incorporating by reference all of the limitations of that claim, wherein said driver includes a centerpiece and a plurality of fins extending generally radially from said centerpiece; wherein each fin is substantially aligned with a corresponding said tine of said staple.

11. The medical device of claim 10, incorporating by reference all of the limitations of that claim, further comprising at least one splay arm including a generally longitudinally extending strut; wherein at least one said splay arm includes a trough extending inward from said strut and substantially longitudinally aligned with a portion of said centerpiece of said driver between two adjacent said fins, whereby relative motion of said driver and at least one said splay arm causes said trough to ride up onto said centerpiece between adjacent said fins such that the distal end of said splay arm moves radially outward.

12. The medical device of claim 11, incorporating by reference all of the limitations of that claim, further comprising at least one notch in said driver, wherein each said notch is substantially aligned with a corresponding trough of a corresponding said splay arm.

13. The medical device of claim 11, incorporating by reference all of the limitations of that claim, wherein at least one said splay arm includes a proximal crossbar located substantially at the proximal end of said strut and oriented at an angle to said strut, and a distal crossbar located substantially at the distal end of said strut and oriented at an angle to said strut.

14. The medical device of claim 13, incorporating by reference all of the limitations of that claim, further comprising a generally tubular tip sleeve within which at least part of each of said staple, said driver, said shuttle and said splay arm are positioned; wherein at least one tab extends inward from said tip sleeve, and wherein said tip sleeve includes a plurality of slots defined therein such that said tines of said staple are splayable through and out of said slots.

15. The medical device of claim 14, incorporating by reference all of the limitations of that claim, wherein each said tab is positioned distal to and is substantially aligned with said proximal crossbar of a corresponding said splay arm.

16. The medical device of claim 13, incorporating by reference all of the limitations of that claim, wherein each said distal crossbar is initially located distal to at least one corresponding said tine of said staple.

17. A method for closing an opening in tissue of a patient, comprising:
providing a staple including a plurality of tines, wherein said staple is deformable from an initial configuration to a splayed configuration and then a closed configuration, and a shuttle fixed to said staple, wherein said shuttle includes a weakened area proximal to said shuttle;
moving said staple to a location in proximity to the opening;
plastically deforming said staple to a splayed configuration;
penetrating at least one said tine into tissue in proximity to the opening; and
plastically deforming said staple to a closed configuration, and then fracturing said shuttle at said weakened area, whereby said staple and a fragment of said shuttle fixed to said staple remain in the patient.

18. The method of claim 17, incorporating by reference all of the limitations of that claim, further comprising providing at least one splay arm including a distal crossbar located distal to said staple; wherein said plastically deforming said staple to said splayed configuration is performed by moving at least of said shuttle and at least one said splay arm relative to the other.

19. The method of claim 18, incorporating by reference all of the limitations of that claim, further comprising shuttling said staple distally after said plastically deforming said staple to a splayed configuration and before said penetrating.

20. The method of claim 19, further comprising moving said distal crossbars away from the longitudinal centerline of said shuttle before said shuttling.

21. The method of claim 19, further comprising providing a driver slidable along the surface of said shuttle; wherein said plastically deforming said staple to a closed configuration is performed by moving at least one of said shuttle and said driver relative to one another to urge said driver into contact with said staple.

22. The method of claim 17, incorporating by reference all of the limitations of that claim, wherein said fracturing is performed by applying tensile force to said shuttle.
